(19) United States
(12) Patent Application Publication
(10) Pub. No.: US 2009/0259275 A1
(43) Pub. Date: Oct. 15, 2009

(54) METHODS OF TREATING BRAIN DAMAGES

(76) Inventor: Qi Wan, Reno, NV (US)

Correspondence Address: CONNOLLY BOVE LODGE & HUTZ, LLP, P O BOX 2207, WILMINGTON, DE, 19899, US

(21) Appl. No.: 12/420,853

(22) Filed: Apr. 9, 2009

Related U.S. Application Data
(60) Provisional application No. 61/045,273, filed on Apr. 15, 2008.

(51) Int. Cl. A61N 1/36

(52) U.S. Cl. 607/45

(57) ABSTRACT

Provided herein are methods of repairing, treating, managing or preventing brain damages. In some embodiments, the methods comprise applying a direct current electric field to direct or modulate the migration of NSPCs towards the region of the brain damage. In certain embodiments, the methods comprise administering an electric field between a cerebral ventricle and the meninx, inclusive, of the brain where the brain damage occurs. In other embodiments, the methods comprise activating a membrane protein of NSPCs by a direct current electric field. In further embodiments, the methods comprise interactions of a membrane protein in NSPC with Rac1, Tiam1, Pak1, and actin cytoskeleton in a protein complex in the presence of an electric field. In still further embodiments, the methods comprise applying an electric field to promote neurogenesis in the subventricular zone or subgranular zone of the brain.

### FIELD OF THE INVENTION

Provided herein are methods of repairing, treating, managing or preventing brain damages, particularly methods comprising the use of an electrical field to direct, modulate or regulate the migration of neural stem cells and progenitor cells (NSPCs) towards regions of the brain damages. In some embodiments, the methods comprise using an electrical field generated by a direct current to direct NSPC migration to replace cell loss in the damaged brain regions.

### BACKGROUND OF THE INVENTION

Stem cell therapies have been used to treat a variety of diseases because stem cells can be grown and transformed into specialized cells with characteristics consistent with cells of various tissues such as muscles or nerves through cell culture. For example, they have been used for treating neurodegenerative diseases, autoimmune diseases, cerebral palsy, diabetes type 2, heart failure, multiple sclerosis, osteoarthritis and degenerative diseases, joint disease, Parkinson's disease, rheumatoid arthritis, stroke and the like.

Many common neurodegenerative diseases, such as Parkinson's disease, stroke and multiple sclerosis, can be caused by a loss of neurons and/or glial cells. It is believed that neurons and glial cells can be generated from neural stem cells or progenitor cells (NSPCs). Therefore, there is a need for methods of directing the migration of NSPCs to the damaged areas so that the lost neurons and glial cells can be replaced or replenished. Further, there is need for methods of repairing, treating, managing or preventing a brain damage by directing or modulating the migration of NSPCs towards regions of brain damages.

The migration of NSPCs is essential not only for early neural development, but also for the functioning of the mature central nervous system (CNS) in both physiological and pathological conditions. Pathological insults such as cerebral ischemia not only stimulate increased generation of endogenous NSPCs in the subventricular zone (SVZ) but also induce migration of the NSPCs to the damaged brain regions or areas. However, only a portion of the newly generated NSPCs is found to migrate to the damaged brain areas. Therefore, there is a need for methods of guiding and speeding up the migration of NSPCs to the damaged areas so that the brain damage can recover faster.

### SUMMARY OF THE INVENTION

Provided herein are methods of repairing, treating, managing or preventing brain damages by directing or modulating the migration of NSPCs towards regions of brain damages. Also provided herein are methods of speeding up the migration of NSPCs to the damaged areas so that the brain damage can recover faster.

In one aspect, provided herein are methods of repairing, treating, managing or preventing a brain damage in a brain of a mammal, such as human, wherein the methods comprise applying a direct current electric field to direct or modulate the migration of one or more NSPCs towards at least a portion of the region of the brain damage.

In some embodiments, the direct current electric field is between a cathode and an anode. In other embodiments, the magnitude of the direct current varies in time. In certain embodiments, the cathode is at or near the region of the brain damage. In other embodiments, the cathode is placed at the skull near the region of the brain damage. In further embodiments, the anode is placed at or near the subventricular zone. In further embodiments, the anode is placed at or near the subgranular zone. In still further embodiments, the anode is placed at or near a cerebral ventricle. In further embodiments, the anode is placed in the inside of a mouth or nasal opening.

In certain embodiments, the NSPCs migrate from the subventricular zone to the region of the brain damage. In other embodiments, the NSPCs migrate from the subgranular zone to the region of the brain damage.

In another aspect, provided herein are methods of repairing, treating, managing or preventing a brain damage in a brain of a mammal, such as human, wherein the methods comprise administering an electric field between a cerebral ventricle and the meninx, inclusive, of the brain. In some embodiments, the electric field is a direct current electric field, a pulsed direct current electric field, an alternative current electric field, a capacitatively coupled electric field (CCEF), or an electric field induced by a pulsed magnetic field. In other embodiments, the electric field is a direct current electric field between an anode at or near the cerebral ventricle and a cathode at or near the meninx. In further embodiments, the meninx is the dura mater, arachnoid mater or pia mater of the damaged brain. In still further other embodiments, the cerebral ventricle is a lateral ventricle, the third ventricle or the fourth ventricle of the damaged brain.

In another aspect, provided herein are methods of repairing, treating, managing or preventing a brain damage in a brain of a mammal, such as human, wherein the methods comprise activating a membrane protein of neural stem cell or progenitor cell by a direct current electric field. In some embodiments, the membrane protein is a NMDA receptor.

In another aspect, provided herein are methods of repairing, treating, managing or preventing a brain damage in a brain of a mammal, such as human, wherein the methods comprise interacting a membrane protein of neural stem cell or progenitor cell with Rac1, TIAM1, PAK1, and actin cytoskeleton to form a protein complex in the presence of an electric field. In certain embodiments, the electric field is a direct current electric field, a pulsed direct current electric field, an alternative current electric field, a capacitatively coupled electric field (CCEF), or an electric field induced by a pulsed magnetic field.

In another aspect, provided herein are methods of repairing, treating, managing or preventing a brain damage in a brain of a mammal, the method comprising applying an electric field to promote neurogenesis in the subventricular zone or subgranular zone of the brain.

In some embodiments, the direct current or pulsed direct current disclosed herein is provided by a power system comprising a battery and a resistor. In other embodiments, the battery has a voltage from about 0.1 volts to about 36 volts, from about 0.25 volts to about 25 volts or from about 0.5 volts to about 15 volts. In further embodiments, the resistor has an electrical resistance from about 1 ohm to about 100 megaohms.

In certain embodiments, the brain damage disclosed herein is a traumatic brain injury, non-traumatic brain injury or neurodegenerative disease. In other embodiments, the brain damage is a non-traumatic brain injury. In further embodiments, the non-traumatic brain injury is stroke, meningitis, hypoxia or anoxia.

In some embodiments, the brain damage is a neurodegenerative disease. In other embodiments, the neurodegenerative disease is Alexander's disease, Alper's disease, Alzheimer's disease, Amyotrophic lateral sclerosis, Ataxia telangiectasia, Batten disease, Bovine spongiform encephalopathy, Canavan disease, Cockayne syndrome, Corticobasal degeneration, Creutzfeldt-Jakob disease, Huntington's disease, AIDS dementia complex, Kennedy's disease, Krabbe's disease, dementia with lewy bodies, Machado-Joseph disease, Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Lichtheim's disease, Schizophrenia, Spinocerebellar ataxia, Spinal muscular atrophy, Steele-Richardson-Olszewski disease or Tabes dorsalis.

In certain embodiments, the methods disclosed herein further comprise applying a pulsed magnetic field to the region of the brain damage. In other embodiments, the methods disclosed herein her comprise applying a second electric field to direct or modulate the migration of one or more neural stem cells or progenitor cells towards at least a portion of the region of the brain damage. In further embodiments, the second electric field is a direct current electric field, a pulsed direct current electric field, or an alternative current electric field.

### BRIEF DESCRIPTION OF FIGURES

FIGS. 1A and 1B depict the migration of neural stem/progenitor cells (NSPCs) in the explant cultures of rat embryonic lateral ganglionic eminence (LGE). FIG. 1A depicts that the explants exhibited a generally circular appearance, with essentially no cell migration out of the periphery after post-plating for 1 hour. FIG. 1B depicts that after 10 hours of post-plating, a symmetrical migration of NSPC cells out of the explant was observed. FIGS. 1C-F depict an embodiment showing that the majority of the cells migrating away from the explant are immature, neural stem/progenitor cells as verified triple staining. FIG. 1C depicts staining by nestin, FIG. 1D depicts staining by DCX (doublecortin), FIG. 1E depicts staining by DAPI (4′,6-diamidino-2-phenylindole), and FIG. 1F depicts the overlap of the nestin, DCX and DAPI stains at 40× (lower images) and 10× magnifications (upper images). The 40× images of FIGS. 1C-F correspond to the boxed areas in the 10× images.

FIGS. 2A-F depicts the effect of using a cathode and an anode to generate a physiological electric field to direct the migration of NSPCs towards the cathode. FIG. 2A depicts the radial migration of NSPCs in lateral ganglionic eminence (LGE) explant culture after 10 hours of post-plating without exposing to an electric field (“Control”). FIG. 2B depicts the exposure of an LGE explant culture to an electric field (30 mV/mm) for 10 hours (“EF Sample”). The exposure to the electric field resulted in an asymmetrical distribution with a higher number of cells in the cathodal side (left) of the explant than that in the anodal side (right). FIG. 2C depicts a diagram illustrating the division of quadrants around a LGE explant. FIG. 2D depicts the effect of the electric field which was expressed as a ratio of cell counts in the cathode-facing quadrant divided by the sum of cell counts in both the cathode and anode-facing quadrants. The white bar shows the cell count ratio for the Control where the total cell count, n, is 226. The black bar shows the cell count ratio for the EF Sample where n is 205 (*p<0.05).

FIGS. 3A and 3B depicts the effect of using a cathode and an anode to generate a physiological electric field to direct and speed up the migration of NSPCs towards the cathode. FIG. 3A depicts directedness of cell migration toward the cathode in cathode-facing quadrants as a function of electric field strength where n is 256, 96, 96 and 96 at 0 mV, 50 mV, 100 mV and 250 mV respectively. (*p<0.05, compared with 0 mV). The data show that the directedness (expressed as a function of cosine) of cell migration toward the cathode depends on the electric field strength. FIG. 3B depicts the velocity of NSPCs moving toward the cathode as a function of electric field strength where n is 256, 96, 96 and 96 at 0 mV, 50 mV, 100 mV and 250 mV respectively. (*p<0.05, compared with 0 mV). The data show that the electric field can increase the velocity of the migration of NSPCs towards cathode in an electric field strength-dependent manner.

FIGS. 4A-D depicts another embodiment showing that the majority of the cells migrating away from the explant are immature, neural stem/progenitor cells as verified triple staining. FIG. 4A depicts staining by nestin, FIG. 4B depicts staining by DCX, FIG. 4C depicts staining by DAPI, and FIG. 4D depicts the overlap of the nestin, DCX and DAPI stains in 40× (lower images) and 10× (upper images) magnifications. The 40× magnification images of FIGS. 4A-D correspond to the boxed areas in the 10× images.

FIGS. 5A-D depict that activation of NMDA receptors (NMDARs) by electric field stimulation can mediate the migration of NSPCs guided by electric field. FIGS. 5A and 5C respectively depict that NR1 and NR2B subunits of NMDARs are expressed on NSPCs migrating cathodally. FIGS. 5B and 5D are higher-magnification images correspond to the boxed areas of FIGS. 5A and 5C. FIGS. 5E-H depict that NMDAR antagonist DAPV (10 μM) significantly attenuated the migration of NSPCs toward the cathode in LGE explants under an electric field at 30 mV/mm after 10 hours of post-plating. FIG. 5E is the Control without exposure to an electric field. FIG. 5F is DAPV without exposure to an electric field. FIG. 5G is the Control with exposure to an electric field. FIG. 5H is DAPV with exposure to an electric field.

FIG. 6 depicts summarized data showing effects of NMDAR inhibition on electric field-directed NSPC migration (Control, n=192; DAPV, n=175; EF, n=187; DAPV+EF, n=212; *p<0.05, EF vs. control or DAPV; #p<0.05, DAPV+EF vs. EF).

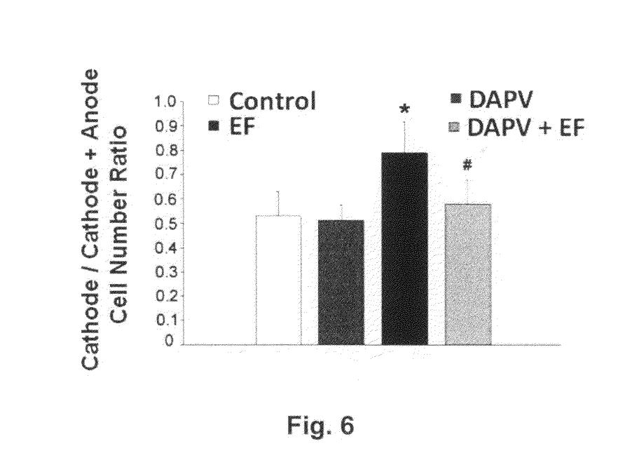

FIGS. 7A-D show that electric field stimulation can promote a physical association of NMDARs with Rac1 signals. FIG. 7A shows that an electric field exposure at 250 mV/mm for 60 minutes significantly enhanced an association of NMDAR NR2B subunit with the specific Rac1 activator Tiam1 and the increased association was suppressed by inhibition of NMDARs with DAPV (10 μM) in cultured LGE explants. FIG. 7B shows summarized data indicating that electric field-induced increase of NR2B-Tiam1 association is dependent on NMDAR activation (right; n=3; *p<0.05, EF vs. control; #p<0.05, DAPV+EF vs. EF). FIG. 7C shows that EF stimulation enhanced the association of phosphorylated PAK1 (p-Pak1) with Tiam1 and the increased association of p-Pak1 with TIAM1 was abolished by inhibition of NMDARs. FIG. 7D shows summarized data indicating that electric field-induced increase of p-Pak1 association with Tiam1 is dependent on NMDAR activation (right; n=3; *p<0.05, EF vs. control; #p<0.05, DAPV+EF vs. EF).

FIGS. 8A and 8B depict electric field stimulation can increase the phosphorylation levels of Pak1. FIG. 8A shows that electric field treatment significantly increased the level of Pak1 phosphorylation (p-Pak1) whereas the electric field-induced increase in the phosphorylation level of Pak1 was blocked by the NMDAR antagonist DAPV. FIG. 8B depicts summarized data showing that the electric field-induced increase of Pak1 phosphorylation requires NMDAR activation (n=3; *p<0.05, EF vs. control; #p<0.05, DAPV+EF vs. EF).

FIGS. 9A and 9B depict electric field-induced NMDAR activation leads to an enhanced association of Rac1 activator Tiam1 with actin cytoskeleton. FIG. 9A shows that electric field exposure increased an association of actin with Tiam1 and the increased actin-Tiam1 association was inhibited by the NMDAR antagonist DAPV. FIG. 9B depicts summarized data showing that electric field-induced increase of actin-Tiam1 association is dependent on NMDAR activation (n=3; *p<0.05, EF vs. control; #p<0.05, DAPV+EF vs. EF).

FIG. 10 depicts an embodiment of a setup for the method of using electric field to repair brain damages in ischemic brain in vivo. It is for in vivo study in rat model of forebrain cerebral stroke. The power supply comprises a 1.4 V hearing aid battery (5) and a 1-20 MΩ resistor (7) in series with the anode. The anodal electrode (6) will be inserted, via a trephination opening (3) in a stroke area or infarct area (2), at 1 mm lateral from the midline (8) into the lateral ventricle (1) at a depth of 4.0 mm. The cathodal electrode is placed on the surface of a stroke area or infarct area (2).

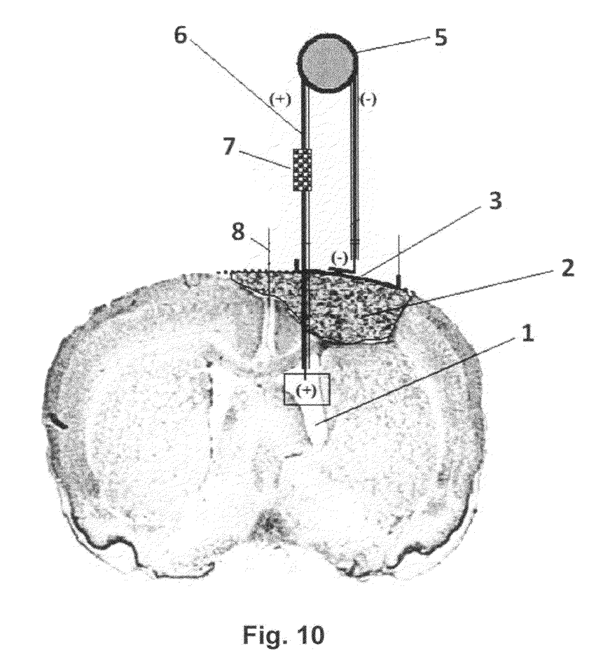

FIG. 11A depicts an electric field stimulation device for the method disclosed herein. FIGS. 11B and 11C depict the placements or locations (1 and 2) of an electrical field stimulation device in a rat.

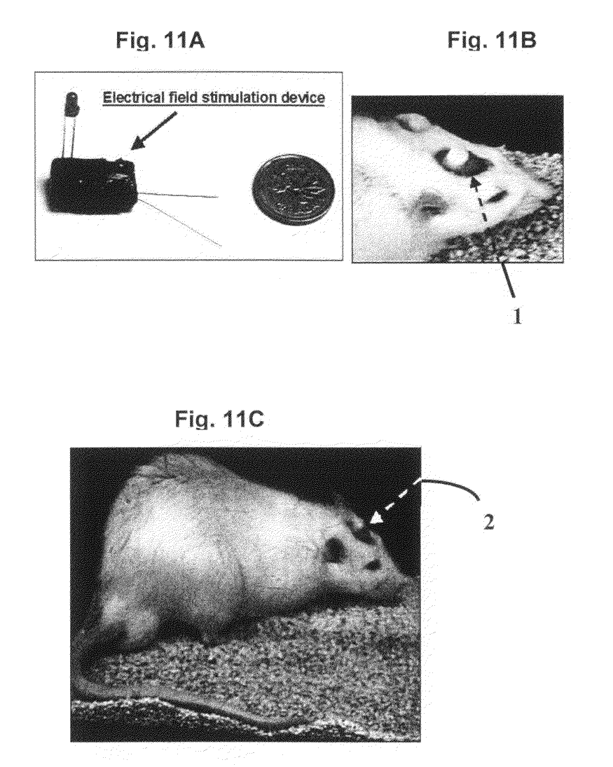

FIG. 12 depicts a graph showing that electrical field can reverse impairments in motor function in rat model of forebrain cerebral stroke. The data show that motor deficits recover after electrical field stimulation at 6 weeks after stroke (n=6 for each group; Data are mean+SE; ANOVA test; *P<0.05, compared with control group; #P<0.05, compared with stroke group).

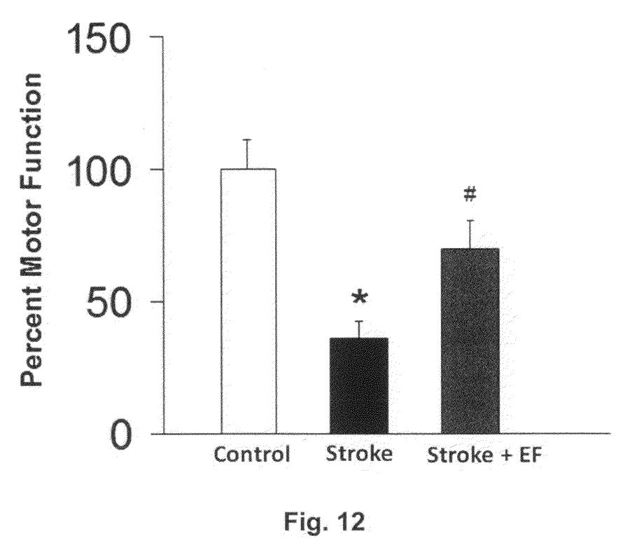

FIGS. 13A and 13B depict the effect of electric field on the migration of NSPCs in cultured organotypic slices of embryonic rat cortex (E17) by local application of 4-chloromethyl benzoyl aminotetramethyl rhodamine (CMTMR), a novel chloromethyl cell tracker, in the cortical ventricular zone (CVZ) at 2 hours after the preparation of the slices. An electric field strength of 20 mV/mm was applied to the slices by placing the anode electrode on the CVZ side and the cathode electrode on the pial side. FIG. 13A shows that the electric field significantly increased the number of migrating NSPCs in the region close to the pial (cathode side) while compared with the number of migrating NSPCs in the control conditions at 12 hours after CMTMR application. FIG. 13A also shows that a reversal of electric field polarity reduced the number of NSPC migration to the pial. FIG. 13B depicts a bar chart showing the mean cell number per slice of the cultured organotypic slices in the absence of an electric field (white bar), in the presence of an applied electric field (black bar), or in the presence of a reversed electric field (the grey bar).

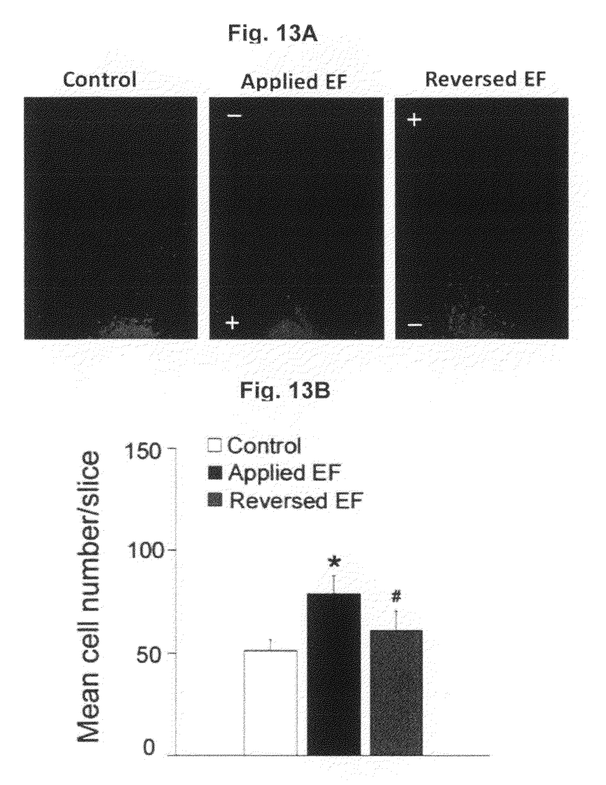

### DEFINITIONS

To facilitate the understanding of the subject matter disclosed herein, a number of terms, abbreviations or other shorthand as used herein are defined below. Any term, abbreviation or shorthand not defined is understood to have the ordinary meaning used by a skilled artisan contemporaneous with the submission of this application.

“Brain damage” refers to traumatic brain injury (TBI) or non-traumatic brain injury. In some embodiments, brain damage disclosed herein also refers to any of the known neurodegenerative diseases including those neurodegenerative diseases disclosed herein.

“Traumatic brain injury” (also known as intracranial injury or head injury) refers to a brain injury which occurs when physical trauma causes brain damage. Traumatic brain injury can result from a closed head injury or a penetrating head injury and is one of two subsets of acquired brain injury (ABI). TBI can cause a host of physical, cognitive, emotional, and social effects. Outcome can be anything from complete recovery to permanent disability or death.

“Non-traumatic brain injury” refers to a brain injury that does not involve external mechanical force (e.g., stroke, meningitis, hypoxia and anoxia). In some embodiments, Brain damage Neurodegenerative disease

“Stroke” (also known as cerebrovascula accident (CVA)) refers to the rapidly developing loss of brain functions due to a disturbance in the blood vessels supplying blood to the brain. This can be due to ischemia caused by thrombosis or embolism, or due to a hemorrhage or heart attack.

“Meningitis” refers to the inflammation of the protective membranes covering the central nervous system, known collectively as the meninges.

“Hypoxia” refers to a pathological condition in which the body as a whole (generalized hypoxia) or region of the body (tissue hypoxia) is deprived of adequate oxygen supply. Brain damages may occur when hypoxia occurs in the brain.

“Anoxia” refers to hypoxia in which there is complete deprivation of oxygen supply. Brain damages may occur when anoxia occurs in the brain.

“Neurogenesis” refers to the process by which neurons are created. Most active during pre-natal development, but it also occurs in adult central nervous system. Neurogenesis is responsible for populating the growing brain.

“Neurodegenerative disease” refers to a condition in which cells of the brain and spinal cord are lost. Neurodegenerative diseases result from deterioration of neurons or their myelin sheath which over time will lead to dysfunction and disabilities. They are may be divided into two groups according to phenotypic effects, although these are not mutually exclusive: (1) conditions causing problems with movements, such as ataxia; and (2) conditions affecting memory and related to dementia. Some non-limiting examples of neurodegenerative diseases include Alexander's disease, Alper's disease, Alzheimer's disease, Amyotrophic lateral sclerosis, Ataxia telangiectasia, Batten disease, Bovine spongiform encephalopathy, Canavan disease, Cockayne syndrome, Corticobasal degeneration, Creutzfeldt-Jakob disease, Huntington's disease, AIDS dementia complex, Kennedy's disease, Krabbe's disease, dementia with lewy bodies, Machado-Joseph disease, Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Lichtheim's disease, Schizophrenia, Spinocerebellar ataxia, Spinal muscular atrophy, Steele-Richardson-Olszewski disease, Tabes dorsalis and the like.

“Alexander's disease” refers to a slowly progressing and fatal neurodegenerative disease. It is a disorder which generally results from a genetic mutation and mostly affects infants and children, causing developmental delay and changes in physical characteristics.

“Alper's disease” (also called Alpers'syndrome, Alpers'Huttenlocher disease, progressive neuronal degeneration of childhood, progressive sclerosing poliodystrophy, and progressive infantile poliodystrophy) refers to a progressive degenerative disease of the central nervous system that occurs in infants and children. It is an autosomal recessive disorder that is sometimes seen in siblings.

“Alzheimer's disease” refers to a degenerative and terminal disease. In its most common form, it occurs in people over 65 years old although a less-prevalent early-onset form also exists. In its early stages, short-term memory loss is the most common symptom, often initially thought to be caused by aging or stress by the sufferer. Later symptoms include confusion, anger, mood swings, language breakdown, long-term memory loss, and the general withdrawal of the sufferer as his or her senses decline. Gradually the sufferer loses minor, and then major bodily functions, until death occurs.

“Amyotrophic lateral sclerosis” refers to a progressive, usually fatal, neurodegenerative disease caused by the degeneration of motor neurons, the nerve cells in the central nervous system that control voluntary muscle movement.

“Ataxia telangiectasia” (also known as AT, Boder-Sedgwick syndrome or Louis-Bar syndrome) refers to a primary immunodeficiency disorder that occurs in an estimated incidence of 1 in 40,000 to 1 in 300,000 births.

“Batten disease” refers to a fatal, autosomal recessive neurodegenerative disorder that begins in childhood. It is a common form of a group of disorders called neuronal ceroid lipofuscinosis (or NCLs).

“Bovine spongiform encephalopathy” refers to a fatal neurodegenerative disease in cattle that causes a spongy degeneration in the brain and spinal cord.

“Canavan disease” refers to an autosomal recessive disorder that causes progressive damage to nerve cells in the brain.

“Cockayne syndrome” refers to an autosomal recessive disorder characterized by growth failure, impaired development of the nervous system, abnormal sensitivity to sunlight (photosensitivity), and premature aging.

“Corticobasal degeneration” refers to a sporadic progressive neurodegenerative disease associated with atrophy of the cerebral cortex and the basal ganglia.

“Creutzfeldt-Jakob disease” refers to a degenerative neurological disorder that is ultimately fatal. Among the types of transmissible spongiform encephalopathy found in humans, it is the most common.

“Huntington's disease” refers to a genetic neurological disorder caused by a trinucleotide repeat expansion in the Huntingtin gene. Huntington's disease's most obvious symptoms are abnormal body movements called chorea and a lack of coordination, but it also affects a number of mental abilities and some aspects of behavior.

“AIDS dementia complex” (also known as HIV dementia, HIV encephalopathy and HIV-associated dementia) refers to a neurological disorder associated with HIV infection and AIDS. Generally, it is a metabolic encephalopathy induced by HIV infection and fueled by immune activation of brain macrophages and microglia.

“Kennedy's disease” (also known as X-linked spinal and bulbar muscular atrophy (SBMA)) refers to is a neuromuscular disease associated with mutations of the androgen receptor (AR). Generally, the androgen receptor gene that is mutated in Kennedy's disease is located on the X chromosome, and the effects of the mutation may be androgen-dependent.

“Krabbe's disease” (also known as globoid cell leukodystrophy or galactosylceramide lipidosis) refers to a degenerative disorder that affects the myelin sheath of the nervous system. This condition is generally inherited in an autosomal recessive pattern.

“Dementia with lewy bodies” refers to the second most frequent cause of hospitalization for dementia, after Alzheimer's disease. Generally, it is characterized by development of abnormal proteinaceous (alpha-synuclein) cytoplasmic inclusions, called lewy bodies, throughout the brain.

“Spinocerebellar ataxia” (SCA) refers to one of a group of genetic disorders characterized by slowly progressive incoordination of gait and often associated with poor coordination of hands, speech, and eye movements. SCA generally has multiple types, each of which could be considered a disease in its own right.

“Machado-Joseph disease” (also known as Spinocerebellar ataxia type 3) refers to a type of spinocerebellar ataxia caused by a mutation in the ATXN3 gene.

“Multiple sclerosis” (also known as disseminated sclerosis or encephalomyelitis disseminata) refers to an autoimmune condition in which the immune system attacks the central nervous system (CNS), leading to demyelination.

“Multiple System Atrophy” (MSA) refers to a degenerative neurological disorder associated with the degeneration of nerve cells in specific areas of the brain. MSA generally is characterized by a combination of the following: (a) Progressive damage to the autonomic nervous system, commonly leading to low blood pressure upon standing, difficulty urinating, and/or abnormal breathing during sleep; (b) Muscle rigidity+/tremor and slow movement (Parkinsonism); and (c) Poor coordination/unsteady walking (ataxia).

“Narcolepsy” refers to a neurological condition most characterized by Excessive Daytime Sleepiness (EDS). Generally, a narcoleptic may experience disturbed nocturnal sleep, which is often confused with insomnia and disorder of Rapid Eye Movement (REM) sleep.

“Borrelia” refers to a genus of bacteria of the spirochete class. Generally, it is a zoonotic, vector-borne disease transmitted primarily by ticks and some by lice, depending on the species. There are at least 37 known species of Borrelia.

“Lyme disease” or “borreliosis” refers to an infectious disease caused by at least three species of bacteria from the genus Borrelia. The vector of infection is typically the bite of an infected black-legged or deer tick, but other carriers, such as ticks in the genus Ixodes, have been implicated.

“Neuroborreliosis” refers to a disease of the central nervous system caused by infection with a spirochete of the genus Borrelia. In some embodiments, it is a late stage of Lyme disease typically involving the skin, joints, and central nervous system.

“Parkinson's disease” refers to a neurodegenerative disorder characterized by a progressive neuronal loss affecting preferentially the dopaminergic neurons of the nigrostriatal projection.

“Pelizaeus-Merzbacher disease” refers to a group of genetic disorders called the leukodystrophies that affect growth of the myelin sheath, the fatty covering on nerve fibers in the brain. It may be caused by a usually recessive mutation of the gene on the long arm of the X-chromosome that codes for a myelin protein called proteolipid protein 1 or PLP1. There are several forms of Pelizaeus-Merzbacher disease including classic, connatal, transitional, adult variants.

“Pick's disease” (also known as Pick disease and PiD) refers to a fronto-temporal neurodegenerative disease that causes slowly worsening decline of mental abilities. PiD generally affects a person's ability to use and understand spoken, written, and even signed language. It may also affect personality, emotions, and social behavior. When the decline in mental abilities is severe enough to interfere with a person's ability to carry out everyday activities, it is called dementia.

“Primary lateral sclerosis” (PLS) refers to a neuromuscular disease characterized by progressive muscle weakness in the voluntary muscles. PLS generally belongs to a group of disorders known as motor neuron diseases. Motor neuron diseases may develop when the nerve cells that control voluntary muscle movement degenerate and die, causing weakness in the muscles they control.

“Prion” is short for proteinaceous infectious particle which refers to a poorly-understood hypothetical infectious agent. It is believed that prions can cause a number of diseases in a variety of mammals, including bovine spongiform encephalopathy (BSE, also known as “mad cow disease”) in cattle and the Creutzfeldt-Jakob disease (CJD) in humans.

“Prion diseases” (also known as Transmissible spongiform encephalopathies (TSEs)) refers to a neurodegenerative disease caused by prions within the central nervous system to form plaques known as amyloids, which disrupt the normal tissue structure. This disruption may be characterized by “holes” in the tissue with resultant spongy architecture due to the vacuole formation in the neurons.

“Refsum's disease” refers to neurological disease that results in the malformation of myelin sheaths around nerve cells. It is a peroxisomal disorder. Refsum's disease may be caused by faulty enzymes during the alpha-oxidation of phytanic acid resulting in buildup of phytanic acid and its unsaturated fatty acid derivatives in the plasma and tissues.

“Sandhoff's disease” refers to an autosomal recessive lipid storage disorder that causes progressive destruction of nerve cells in the brain and spinal cord. Sandhoff disease may be caused by mutations in the HEXB gene. The HEXB gene provides instructions for making a protein that is part of two critical enzymes in the nervous system. These enzymes, beta-hexosaminidase A and beta-hexosaminidase B, function in nerve cells to break down fatty substances, complex sugars, and molecules that are linked to sugars. In particular, beta-hexosaminidase A breaks down a fatty compound called GM2 ganglioside. Mutations in the HEXB gene disrupt the activity of these enzymes, preventing the breakdown of GM2 ganglioside and other molecules.

“Schilder's disease” (also known as diffuse myelinoclastic sclerosis) refers to a neurodegenerative disease that presents clinically as pseudotumoural demyelinating lesions. It may present adrenal atrophy and diffuse cerebral demyelination. It generally begins in childhood, affecting children between 5 and 14 years old.

“Pernicious anemia” (also known as Biermer's anaemia or Addison's anaemia or Addison-Biermer anaemia) refers to a form of megaloblastic anemia due to vitamin B₁₂ deficiency because of impaired absorption of vitamin B₁₂ due to the absence of intrinsic factor in the setting of atrophic gastritis, or the loss of gastric parietal cells.

“Subacute combined degeneration of spinal cord secondary to Pernicious Anaemia” (also known as Lichtheim's disease) refers to degeneration of the posterior and lateral columns of the spinal cord as a result of vitamin B₁₂ deficiency. It is usually associated with pernicious anemia.

“Schizophrenia” refers to a psychiatric diagnosis that describes a mental illness characterized by impairments in the perception or expression of reality, most commonly manifesting as auditory hallucinations, paranoid or bizarre delusions or disorganized speech and thinking in the context of significant social or occupational dysfunction.

“Spinocerebellar ataxia” refers to a group of genetic disorders characterized by slowly progressive incoordination of gait and often associated with poor coordination of hands, speech, and eye movements. In some embodiments, atrophy of the cerebellum occurs.

“Spinal muscular atrophy” refers to a group of different disorders, all having in common a genetic cause and the manifestation of weakness due to loss of the motor neurons of the spinal cord and brainstem.

“Steele-Richardson-Olszewski disease” (also known as Progressive supranuclear palsy (PSP)) refers to a rare degenerative disorder involving the gradual deterioration and death of selected areas of the brain. The initial symptom may be loss of balance and falls, and changes in personality, general slowing of movement, and visual symptoms. Later symptoms and signs may be dementia.

“Tabes dorsalis” refers to a slow degeneration of the nerve cells and nerve fibers that carry sensory information to the brain. The degenerating nerves may be in the dorsal columns of the spinal cord and may carry information that help maintain a person's sense of position.

“Autoimmune diseases” refers to any disease that arises from the failure of an organism to recognize its own constituent parts (down to the sub-molecular levels) as self, which results in an immune response against its own cells and tissues.

“Cerebral palsy” refers to a group of non-progressive, non-contagious conditions that cause physical disability in human development. It can be divided into four major classifications to describe the different movement impairments. These classifications reflect the area of brain damaged. The four major classifications are spastic, athetoid/dyskinetic, ataxic and mixed cerebral palsy.

“Diabetes type 2” (also known as diabetes mellitus type 2, type 2 diabetes, non-insulin-dependent diabetes mellitus (NIDDM), or adult-onset diabetes) refers to a metabolic disorder that is primarily characterized by insulin resistance, relative insulin deficiency and hyperglycemia.

“Heart failure” (also known as congestive heart failure (CHF), congestive cardiac failure (CCF)) refers to a condition that can result from any structural or functional cardiac disorder that impairs the ability of the heart to fill with or pump a sufficient amount of blood through the body.

“Osteoarthritis” (also known as degenerative arthritis or degenerative joint disease) refers to a condition in which low-grade inflammation results in pain in the joints, caused by abnormal wearing of the cartilage that covers and acts as a cushion inside joints and destruction or decrease of synovial fluid that lubricates those joints.

“Rheumatoid arthritis” refers to a chronic, inflammatory autoimmune disorder that causes the immune system to attack the joints or other organs as well.

“Neuron” refers to an electrically excitable cell in the nervous system that can process and transmit information. Neuron can be found in the brain, peripheral nerves, and spinal cord in vertebrates and ventral nerve cord in invertebrates.

“Glial cell” (also known as neuroglia or glia) refers to non-neuronal cell that provides support and nutrition, maintains homeostasis, forms myelin, and participates in signal transmission in the nervous system.

“Stem cell” refers to a cell that is capable of retaining the ability to reinvigorate themselves through mitotic cell division and can differentiate into a diverse range of specialized cell types. Stem cells can be found in most multi-cellular organisms. The two broad types of mammalian stem cells are embryonic stem cells that are found in blastocysts, and adult stem cells that are found in adult tissues.

“Progenitor cell” refers to immature or undifferentiated cells, typically found in post-natal animals. Like stem cells, progenitor cells have a capacity for self-renewal and differentiation, but these properties may be more limited than stem cells.

“Subventricular zone” (SVZ) refers to a paired brain structure situated throughout the lateral walls of the lateral ventricles. SVZ can serve as a source of NSPCs in the process of adult neurogenesis.

“Subgranular zone” (SGZ) refers to a brain region in the dentate gyrus where adult neurogenesis occurs. SGZ generally lays deep within the hippocampal parenchyma, at the interface between the granule cell layer and the hilus of the dentate gyrus. SGZ can serve as a source of NSPCs in the process of adult neurogenesis.

“NMDA” refers to N-methyl-D-aspartic acid which is an amino acid derivative acting as a specific agonist at the NMDA receptor, and therefore mimics the action of the neurotransmitter glutamate on that receptor.

“NMDA receptor” (NMDAR) refers to an ionotropic receptor for glutamate. Activation of NMDA receptors generally results in the opening of an ion channel that is nonselective to cations. This allows the flowing of Na⁺ and small amounts of Ca²⁺ ions into the cell and K⁺ out of the cell. In some embodiments, calcium flux through NMDARs play a critical role in synaptic plasticity, a cellular mechanism for learning and memory.

“Ion channel” refers to pore-forming proteins that help to establish and control the small voltage gradient across the plasma membrane of all living cells by allowing the flow of ions down their electrochemical gradient. They are present in the membranes that surround all biological cells.

“Rac1” or “Ras-related C3 botulinum toxin substrate 1” refers to a small signaling G protein (e.g., a GTPase), and is a member of the Rac subfamily of the family Rho family of GTPases. Rac1 is encoded by the gene RAC1. In some embodiments, Rac1 is a pleiotropic regulator of many cellular processes, including the cell cycle, cell-cell adhesion, motility (through the actin network), and of epithelial differentiation.

“Tiam1” or “T-cell lymphoma invasion and metastasis-inducing protein 1” refers to a human protein gene that can modulate the activity of Rho GTP-binding proteins and connects extracellular signals to cytoskeletal activities. In some embodiments, TIAM1 acts as a GDP-dissociation stimulator protein that can stimulate the GDP-GTP exchange activity of Rho-like GTPases and activate them. In some embodiments, TIAM1 activates RAC1, CDC42, and to a lesser extent RHOA.

“Pak1” or “P21/Cdc42/Rac1-activated kinase 1” refers to a human gene that is one of the effectors that link RhoGTPases to cytoskeleton reorganization and nuclear signaling. Pak1 belongs to the PAK protein family which includes Pak1, Pak2, Pak3 and Pak4. These PAK proteins can serve as targets for the small GTP binding proteins Cdc42 and Rac and have been implicated in a wide range of biological activities. Pak1 can regulate cell motility and morphology.

“Actin” refers to a globular found in most eukaryotic cells. Actin is the monomeric subunit of microfilaments, one of the three major components of the cytoskeleton, and of thin filaments, which are part of the contractile apparatus in muscle cells. Actin can participate in many important cellular functions such as muscle contraction, cell motility, cell division and cytokinesis, vesicle and organelle movement, cell signaling, and the establishment and maintenance of cell junctions and cell shape.

“Activating,” “activate” or “activation” refers to the opening of an ion channel or a cell membrane, i.e., the conformational change that allows ions to pass through the ion channel or cell membrane.

“Repairing” and “repair” refers to an action that occurs while a patient is suffering damages arising from a specified disease or disorder, which restores damaged parts or regions to sound condition after damage or injury

“Treating,” “treat” and “treatment” refers to an action that occurs while a patient is suffering from a specified disease or disorder, which reduces the severity or symptoms of the disease or disorder or retards or slows the progression or symptoms of the disease or disorder.

“Preventing,” “prevent” and “prevention” refers to an action that occurs before a patient begins to suffer from a specified disease or disorder, which inhibits or reduces the severity or symptoms of the disease or disorder.

“Managing,” “manage” and “management” encompass preventing the recurrence of a specified disease or disorder in a patient who has already suffered from the disease or disorder and/or lengthening the time that a patient who has suffered from the disease or disorder remains in remission. The terms encompass modulating the threshold, development and/or duration of the disease or disorder or changing the way that a patient responds to the disease or disorder.

“Direct current” (DC) refers to the unidirectional flow of electric charge. The magnitude of the direct current used herein can be constant with time or in a time-varying waveform such as sine waves, triangular waves and square waves.

“Alternating current” (AC) refers to an electrical current whose magnitude and direction vary with time. The magnitude and/or direction of alternating currents may be in different waveforms such as sine waves, triangular waves and square waves.

In the following description, all numbers disclosed herein are approximate values, regardless whether the word “about” or “approximate” is used in connection therewith. They may vary by 1 percent, 2 percent, 5 percent, or, sometimes, 10 to 20 percent. Whenever a numerical range with a lower limit, R^{L} and an upper limit, R^{U}, is disclosed, any number falling within the range is specifically disclosed. In particular, the following numbers within the range are specifically disclosed: R=R^{L}+k*(R^{U}−R^{L}), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed.

### DETAILED DESCRIPTION OF THE INVENTION

Provided herein are methods of repairing, treating, managing or preventing brain damages in brains of mammals such as human. In some embodiments, the methods disclosed herein comprise applying a direct current electric field to direct or modulate the migration of one or more neural stem cells and progenitor cells (NSPCS) towards at least a portion of the region of the brain damage.

The direct current electric field can be applied in any manner known to a skilled artisan. The direct current used herein can be produced by any conventional sources such as batteries, solar cells, fuel cells, thermocouples, and commutator-type electric machines of the dynamo type. In some embodiments, the direct current used herein can also be obtained by converting from an alternating current using a rectifier or AC to DC converter. The magnitude of the direct current used herein can be constant with time or in a time-varying waveform such as sine waves, triangular waves and square waves. In certain embodiments, the magnitude of the direct current varies in time. In other embodiments, the magnitude of the direct current is in a sine, triangular or square waveform where the flow of the electric charge is kept unidirectional or in one direction.

In some embodiments, the polarity of the DC electric field or the direction of the migration of NSPCs may be changed. In other embodiments, the polarity of the DC electric field or the direction of the migration of NSPCs is changed at least once in a fixed time period from about 1 second to about 6 hours, from about 10 seconds to about 3 hours, from about 20 seconds to about 1 hour, from about 30 seconds to about 45 minutes, or from about 1 minute to about 30 minutes.

The direct current electric field can be applied or maintained between two or more applicators or electrodes. In some embodiments, the direct current electric field is applied between a cathode and an anode. In certain embodiments, the cathode is at or near the region of the brain damage where the brain damage has occurred or may occur. The cathode is placed at the skull near the region of the brain damage.

In other embodiments, the anode is placed at a source of NSPCs such as the subventricular zone, the subgranular zone, or the location of the brain where NSPCs are implanted. In some embodiments, the neural stem cells or progenitor cells migrate from the subventricular zone to the region of the brain damage. In other embodiments, the neural stem cells or progenitor cells migrate from the subgranular zone to the region of the brain damage. In some embodiments, no NCPCs are implanted into the brain. In other embodiments, NCPCs are implanted into the brain and the NCPCs are directed to the region of the brain damage by the methods disclosed herein.

In certain embodiments, one or more anodes, or one or more cathodes, or two or more electrodes may be used in circumstances where require more than a cathode and an anode. For example, two or more cathodes may be required when the region of brain damage is too large for one cathode to handle. Similarly, two or more anodes may be required when more than one source of NSPCs is required. Similarly, two or more cathodes and/or two or more anodes may be used to direct the migration of NSPCs to regions, such as the regions of brain damage or the sources of NSPCs, where it is difficult to get access into.

Provided also herein are methods of repairing, treating, managing or preventing a brain damage in a brain of a mammal such as human, wherein the methods comprise administering an electric field between a cerebral ventricle and the meninx, inclusive, of the brain.

In some embodiments, the direct current electric field is applied between an anode and a cathode, wherein the anode is placed at or near the cerebral ventricle of the brain or a part of the cerebral ventricle, and wherein the cathode is placed at or near the meninx of the brain or a part of the meninx. In some embodiments, the cathode is place at the skull near the region of the brain damage. In some embodiments, the anode will be placed in the inside of a mouth or nasal opening.

Generally, the cerebral ventricles are within the ventricular system which is a set of structures in the brain continuous with the central canal of the spinal cord. There are four cerebral ventricles: the pair of lateral ventricles, the third ventricle and the fourth ventricle. The two lateral ventricles, located within the cerebrum, are relatively large and generally C-shaped. It is believed that the successive generation of neurons in the lateral ventricles of the embryo gives rise to the 6-layered structure of the neocortex, constructed from the inside out during development. Each lateral ventricle can extend into the frontal, temporal and occipital lobes via the frontal (anterior), temporal (inferior), and occipital (posterior) horns, respectively. The lateral ventricles can communicate via the interventricular foramina with the third ventricle, which is generally located centrally within the diencephalon. The third ventricle can communicate via the cerebral aqueduct, located within the midbrain, with the fourth ventricle, which is generally located within the hindbrain and continuous with the central canal. In some embodiments, the anode of the direct current electric field is placed at or near a lateral ventricle, the third ventricle or the fourth ventricle.

In some embodiments, the brain damages occur at or near the meninx or a part of the meninx. In certain embodiments, the cathode is placed at or near a part of the meninx, such as dura mater, arachnoid mater or pia mater of the damaged brain. The meninx refers to a system of membranes which envelops the central nervous system. The meninx generally consists of three layers: the dura mater, the arachnoid mater, and the pia mater. The primary function of the meninx and of the cerebrospinal fluid is to protect the central nervous system.

The pia mater generally is a thin and delicate membrane attached to the brain or the spinal cord, and follows all the minor contours of the brain such as gyri and sulci. The pia mater is the meningeal envelope which firmly adheres to the surface of the brain and spinal cord. The pia mater generally comprises fibrous tissue covered on its outer surface by a sheet of flat cells which may be impermeable to fluid. The pia mater can be pierced by blood vessels which travel to the brain and spinal cord, and its capillaries are responsible for nourishing the brain.

The arachnoid mater is in the middle of the meninx and generally has a spider web-like appearance. It can provide a cushioning effect for the central nervous system. The arachnoid mater exists as a thin, transparent membrane. Generally, the arachnoid mater comprises fibrous tissue and, like the pia mater, is covered by flat cells which may be impermeable to fluid. The arachnoid in general does not follow the convolutions of the surface of the brain so that it generally looks like a loosely fitting sac.

The dura mater generally is a thick, durable membrane, closest to the skull and contains larger blood vessels which split into the capilliaries in the pia mater. Generally, the dura mater comprises dense fibrous tissue, and is covered by flattened cells at its inner. The dura mater surrounds and supports the large venous channels (dural sinuses) carrying blood from the brain toward the heart.

In some embodiments, the brain damages occur below the meninx or a part of the meninx such as cerebral cortex. In certain embodiments, the cathode is placed at or near the cerebral cortex or a part of the cerebral cortex such as the grey matter, the white matter, sulci and gyri. The cerebral cortex is a structure within the brain that plays a key role in memory, attention, perceptual awareness, thought, language, and consciousness. The outermost layer of the cerebrum comprises the gray matter which is formed by neurons and their unmyelinated fibers, whereas the white matter below the gray matter of the cortex is formed predominantly by myelinated axons interconnecting different regions of the central nervous system.

The surface of the cerebral cortex is generally folded in large mammals such as human, wherein more than two-thirds of the cortical surface is buried in grooves known as sulci. The phylogenetically most recent part of the cerebral cortex, the neocortex, can be differentiated into six horizontal layers, whereas the more ancient part of the cerebral cortex, the hippocampus (also known as archicortex), has at most three cellular layers, and is divided into subfields. There are layers in the upper part of the cortical grooves known as gyri.

Provided also herein are methods of repairing, treating, managing or preventing brain damages wherein the methods comprise activating a membrane protein of neural stem cell or progenitor cell by a direct current electric field.

Provided also herein are methods of repairing, treating, managing or preventing brain damages wherein the methods comprise interacting a membrane protein of neural stem cell or progenitor cell with Rac1, TIAM1, PAK1, and actin cytoskeleton to form a protein complex in the presence of an electric field.

The electric field may be a direct current (DC) electric field, a pulsed direct current electric field, an alternating current (AC) electric field, a capacitatively coupled electric field (CCEF), or an electric field induced by a pulsed magnetic field. Both the DC and AC electric fields may be modulated by conventional modulation techniques. In general, the migration of the NSPCs depends on, inter alia, the strength of the electric field. Any suitable electric field strength suitable for treating mammals such as human can be used for the methods disclosed herein. The electric field should not be too low so that it is too weak to activate any effect. However, the electric field should not be too high to cause damages to the brain or a part of the brain. In some embodiments, the magnitude of electric field is from about 0.1 mV/mm to about 1000 mV/mm, from about 0.5 mV/mm to about 500 mV/mm, from about 1 mV/mm to about 250 mV/mm, from about 1 mV/mm to about 100 mV/mm, or from about 5 mV/mm to about 50 mV/mm.

The electric field strength may be constant, or varying over time. An electric field strength that is varying over time can be a sinusoidally varying field. In one embodiment, a temporally varying capacitatively coupled field is used. The sinusoidally varying electric field may have a peak voltage across electrodes placed across the cells of from about 1 volt to about 10 volts.

In some embodiments, the electric field is generated by a direct current (DC) disclosed herein. In certain embodiments, the electric field is generated by an alternating current (AC). Depending on the application, the AC or electric field may have a frequency from about 1 hz to about 10 MHz, from about 100 hz to about 1 MHz, from about 1 Khz to about 500 KHz, from about 2 Khz to about 200 KHz or from about 5 Khz to about 100 KHz.

In certain embodiments, the electric field disclosed herein is in pluses. In other embodiments, the pluses are in suitable waveform such as sine, triangualar and square waveform. The duration between pluses may be from about 1 microsecond to about 10 hour, from about 10 microseconds to about 60 minutes, from about 0.1 seconds to about 45 minutes, from about 1 second to about 30 minutes, from about 10 seconds to about 15 minutes, or from about 15 seconds to about 10 minutes.

The electric field may be provided by one or more applicators or electrodes. The applicators or electrodes may have any configuration known to a skilled artisan. In some embodiments, the configuration is in the form of two parallel plates or electrodes. Other non-limiting examples of suitable configurations include stray-field electrodes, resonant cavities or waveguides at higher frequencies. The applicators or electrodes can be placed at or near any region of the brain damages and/or any source of NSPCs. In some embodiments, one or more applicators or electrodes are placed at or near the cerebral ventricle of the brain or a part of the cerebral ventricle. In other embodiments, one or more applicators or electrodes are placed at or near the meninx of the brain or a part of the meninx. In further embodiments, one or more applicators or electrodes are placed at or near the skull near the region of the brain damage.

The anode and the cathode can be made of any chemically inert electrical conductor. Some non-limiting examples of suitable electrical conductors include aluminum, gold, silver, platinum, iridium or an alloy thereof.

The electric field used herein can be produced using any suitable method and apparatus, including such methods and apparatuses known in the art. In some embodiments, the direct current is provided by a power system comprising a battery and a resistor. In other embodiments, the battery has a voltage from about 0.1 volts to about 36 volts, from about 0.25 volts to about 25 volts, from about 0.5 volts to about 15 volts or from about 1 volt to about 10 volts. In further embodiments, the resistor has an electrical resistance from about 1 ohm to about 100 megaohms, from about 2 ohm to about 10 megaohms, from about 5 ohm to about 1 megaohms or from about 10 ohm to about 100 kiloohms.

In certain embodiments, the electric field is generated with the aid of a capacitatively coupling device such as a SpinalPak™ (obtained from EBI, L.P., Parsippany, N.J., U.S.A.) or a DC stimulation device such as an SpF™ XL IIb spinal fusion stimulator (obtained from EBI, L.P.).

The pulsed magnetic field can be produced using any known method and apparatus, such as a single coil or a pair of Helmholtz coils or the EBI Bone Healing System™ Model 1026 (obtained from EBI, L.P.). Any pulse duration, pulse intensity, and numbers of pulses of the pulsed magnetic field known to a skilled artisan can be used. In some embodiments, the pulse duration of the pulsed magnetic field can be from about 10 microseconds per pulse to about 2000 microseconds per pulse, or from about 100 microseconds per pulse to about 500 microseconds per pulse. In one embodiment, pulses are comprised in magnetic bursts. A burst can comprise from one pulse up to about two hundred pulses. In some embodiments, a burst comprises from about ten pulses to about thirty pulses. Bursts can be repeated while applying the pulsed magnetic field to the damaged brain. In some embodiments, bursts can be repeated at a frequency of from about 1 Hertz (Hz) to about 100 Hz, or from about 10 Hz to about 20 Hz. In other embodiments, a burst can have a duration from about 10 microseconds to about 40,000 microseconds, from about 20 microseconds to about 10,000 microseconds, or from about 100 microseconds to about 5,000 microseconds

Any membrane protein known to a skilled artisan can be used for the methods disclosed herein. In some embodiments, the membrane protein is a NMDA receptor. In other embodiments, the NMDA receptor is NMDAR1, NMDAR2A, NMDAR2B, NMDAR2C, NMDAR2D or a combination thereof. Some non-limiting examples of suitable membrane proteins include GABA receptors, glycine receptors, voltage-gated channels, G-protein coupled receptors and receptors for neurotropic factors such as NGF, BDNF, and IGF.

Optionally, the AC electric field or DC electric field used in the methods of repairing, treating, managing or preventing a brain damage disclosed herein can be modulated by conventional modulation techniques to control the destination and/or magnitude of the migration of the NSPCs in the damaged brain. For example, the electric field or direct current electric field can be modulated by varying the amplitude (“intensity”), its phase (“timing”) and its frequency (“pitch”) of the waveform of the AC electric field or DC electric field.

Optionally, the methods of repairing, treating, managing or preventing a brain damage disclosed herein can further comprise the step of applying a second electric field to direct or modulate the migration of one or more neural stem cells or progenitor cells towards at least a portion of the region of the brain damage. The second electric field can be a direct current electric field, an alternative current electric field, a capacitatively coupled electric field (CCEF), or an electric field induced by a pulsed magnetic field.

Optionally, the methods of repairing, treating, managing or preventing a brain damage disclosed herein can further comprise the step of applying a pulsed magnetic field to the region of the brain damage. The pulsed magnetic field can be used to direct or modulate the migration of one or more neural stem cells or progenitor cells towards at least a portion of the region of the brain damage.

The methods disclosed herein can be used to repair, treat, manage or prevent any brain damage known to a skilled artisan. In some embodiments, the brain damage is a traumatic brain injury, non-traumatic brain injury, neurodegenerative disease or a combination thereof. In other embodiments, the brain damage is a traumatic brain injury which is caused by physical trauma to the brain. In other embodiments, the brain damage is a non-traumatic brain injury. Some non-limiting examples of suitable non-traumatic brain injuries include stroke, meningitis, hypoxia and anoxia.

In certain embodiments, the brain damage is a neurodegenerative disease. Some non-limiting examples of suitable neurodegenerative diseases include Alexander's disease, Alper's disease, Alzheimer's disease, Amyotrophic lateral sclerosis, Ataxia telangiectasia, Batten disease, Bovine spongiform encephalopathy, Canavan disease, Cockayne syndrome, Corticobasal degeneration, Creutzfeldt-Jakob disease, Huntington's disease, AIDS dementia complex, Kennedy's disease, Krabbe's disease, dementia with lewy bodies, Machado-Joseph disease, Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Lichtheim's disease, Schizophrenia, Spinocerebellar ataxia, Spinal muscular atrophy, Steele-Richardson-Olszewski disease, Tabes dorsalis and the like.

As demonstrated above, embodiments disclosed herein provide various compounds that can be used for treating, managing or preventing a disease that is related to angiogenesis and other diseases disclosed herein. While this disclosure has been described with respect to a limited number of embodiments, the specific features of one embodiment should not be attributed to other embodiments disclosed herein. No single embodiment is representative of all aspects of this disclosure. In some embodiments, the compositions or methods may include numerous compounds or steps not mentioned herein. In other embodiments, the compositions or methods do not include, or are substantially free of, any compounds or steps not enumerated herein. Variations and modifications from the described embodiments exist. For example, the pharmaceutical compositions disclosed herein need not comprising only the compounds disclosed herein. It can comprise any type of compounds generally suitable for treating, managing or preventing a disease that is related to angiogenesis. It is noted that the methods for making and using the compounds disclosed herein are described with reference to a number of steps. These steps can be practiced in any sequence. One or more steps may be omitted or combined but still achieve substantially the same results. The appended claims intend to cover all such variations and modifications as falling within the scope of this disclosure.

### EXAMPLES

The following examples are intended for illustrative purposes only and do not limit in any way the scope of the present invention.

### Procedure For EF Application and the Imaging of NSPC Migration

Explants (100-300 μm in diameter) were selected for observation. For the electric field (EF) application, agar-salt bridges were used to connect silver/silver chloride electrodes in beakers containing Steinberg's solution to pools of excess culture medium at either side of the chamber. The electric field strengths were measured directly at the beginning and end of the observation period. For time-lapse observation, HEPES acid (25 mM) was added to the culture medium to adjust pH to 7.4. Time-lapse imaging was performed with an inverted microscope (Zeiss Axiovert 200M, obtained from Zeiss, Oberkochen, Germany) that was used to record digitally the migration of NSPCs for 3 hours. The inverted microscope was equipped with an ORCA-ER camera and Uniblitz bright field shutter, allowing acquisition of transmitted phase-contrast or differential interference contrast (DIC) images. Hardware was controlled by Axiovision software (obtained from Zeiss). Images were acquired every ten minutes. For long-term observations, the cultures and EF stimulation device were kept in the CO₂ incubator. The field strength of 30 mV/mm was utilized for all experiments with an exposure time of 10 hours. Exposure was initiated at 2-3 hours post-plating and terminated at 12-13 hours post-plating. At the end of exposure period explants were fixed in 4% paraformaldehyde and then digitally photographed for quantification.

### Procedure For Quantification of Cell Motion

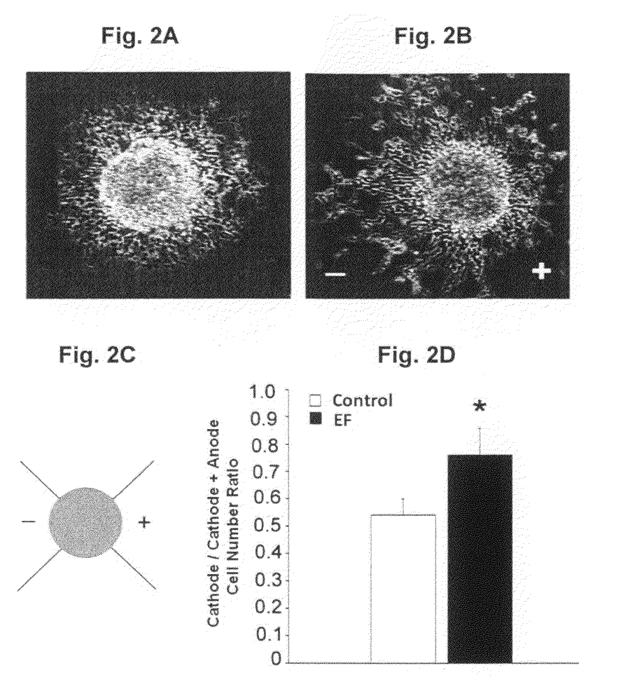

The explant was divided into four diagonal quadrants (as shown in FIG. 2C) for the analysis of cell motion, the results of which are as shown in FIG. 2D. Two of the quadrants were designated as cathode facing and anode facing (see FIG. 2C). To quantify velocity and directedness of cell motion, the cell centroid was calculated with Image J software (obtained from NIH). This yielded (X,Y) coordinates in micrometers. The starting and final positions of cell centroids were exported to Minitab software (obtained from Minitab Inc., State College, Pennsylvania). The absolute motion in the X and Y planes was used to calculate displacement for each cell according to the Pythagoras theorem. Displacement was divided by three (hours) to yield velocity in μm/hr. The directedness of motion was expressed as a function of cosine. It was calculated by defining movements in the X-plane towards the cathode (or mock-cathode) as negative. This was divided by displacement to yield directedness for each cell. Therefore, a cell moving directly towards the cathode would have a value of −1, while a cell moving directly towards the anode would have a value of +1.

For quantification of long-term EF-exposed cells, the symmetry was expressed as a function of the distribution of cells around the explant. Total cell numbers in the cathode and anode facing quadrants were calculated. The data was expressed as a ratio of cell counts in the cathode-facing quadrant divided by the sum of cell counts in both the cathode and anode-facing quadrants. This ratio would be equal to +1 if all cells were located in the cathode quadrant, 0 if all cells were located in the anode quadrant, and 0.5 if cells were evenly distributed.

### Procedure For Immunocytochemistry and Coimmunoprecipitation

The methods for immunocytochemistry and coimmunoprecipitation have been described in detail in Ning et al., “Dual neuroprotective signaling mediated by downregulating two distinct phosphatase activities of PTEN,” J. Neurosci., 24, 4052-60, (2004); and Liu et al., “Ischemic insults direct glutamate receptor subunit 2-lacking AMPA receptors to synaptic sites,” J. Neurosci., 26, 5309-19 (2006), both of which are incorporated herein by reference. For immunocytochemical labeling, the guinea-pig anti-DCX (obtained from Santa Cruz Biotechnology, Santa Cruz, Calif.), mouse anti-nestin (obtained from Chemicon, Billerica, Mass.), rabbit anti-NR1 (obtained from Chemicon), and rabbit anti-NR2B (obtained from Novas Biologicals, Littleton, Colo.) primary antibodies were used. Secondary antibodies consisted of Alex Fluor 488 and 596 were purchased from Molecular Probes, Eugene, Oreg. Imaging was performed with a Zeiss LSM 510 META confocal microscope and image processing was performed with Image J software (obtained from NIH). For coimmunoprecipitation assay, rabbit anti-TIAM1 (obtained from Santa Cruz), rabbit anti-NR2B (obtained from Novus Biologicals), rabbit anti-phosphorylated PAK1 at serine 423 (obtained from Santa Cruz) and mouse anti-actin (obtained from Chemicon) were used.

All data are presented as mean±SEM. Statistical significance was placed at p<0.05. Significance was assessed with the t-test and ANOVA test.

### Example 1

### Explant Cultures of Rat Embryonic LGE

The explant cultures of rat (Wistar) lateral ganglionic eminence (LGE) were prepared from embryonic day 17-18 rats and placed on poly-L-lysine/laminin-coated coverslips in a micro-chamber built for EF application as described in Zhao et al., “Orientation and directed migration of cultured corneal epithelial cells in small electric fields are serum dependent,” J. Cell Sci., 109, 1405-14 (1996), which is incorporated herein by reference. The cultures were placed in a 5% CO₂ incubator for recovery for at least 2 hours before use. The incubation medium comprised minimum essential medium (MEM) supplemented with 10% FBS and 24 mM NaHCO₃.

### Example 2

### EFs at Physiological Strengths Guide and Speed NSPC Migration Towards the Cathode

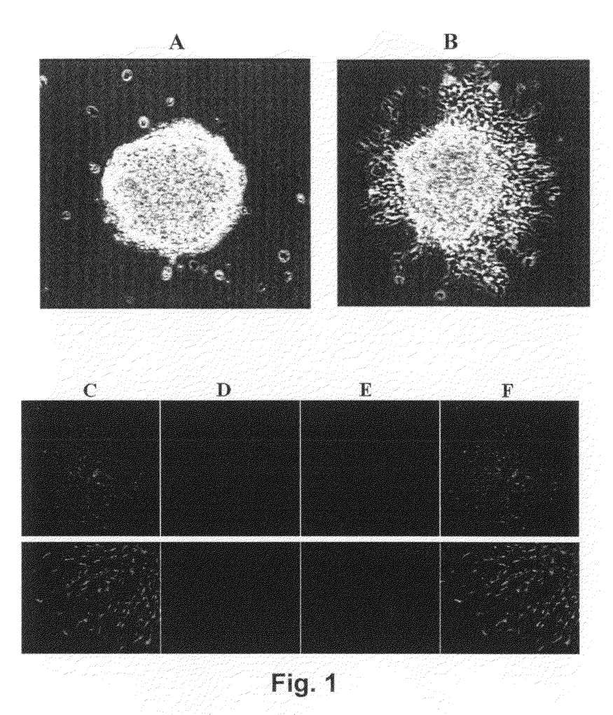

Explant cultures of the LGE from E17-18 rats were used to study the effect of EFs on NSPC migration. In the control cultures, cells moved radially out of the explants and were symmetrically distributed around the circumference of each explant (see FIGS. 1A and 1B). To characterize the phenotypes of cells that migrate out of the explants, immunocytochemical staining with an antibody against nestin (an intermediate filament protein that is typical for undifferentiated NSPCs) and an antibody against doublecortin (DCX, a protein specifically expressed in immature, migrating neurons) was performed. It was found that 76% of the cells migrating out of the explants were nestin-positive cells and 89% of nestin-positive cells were positive for DCX labeling (see FIGS. 1C, 1D and 1E). These data indicate that in some embodiments the majority of cells migrating out of the LGE explants are immature, migrating neurons that are derived from NSPCs.

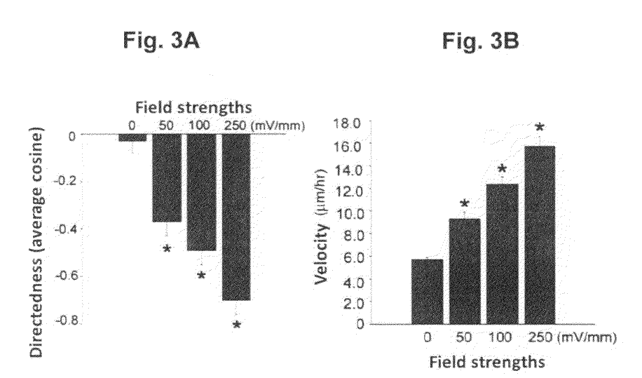

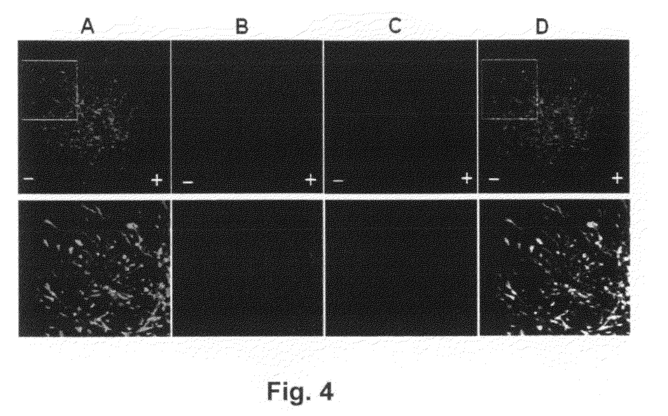

The explant cultures were exposed to EFs at the range of physiologically relevant strengths from about 30 mV/mm to about 250 mV/mm. FIGS. 2A-D and 3A showed that EFs directed the migration of cells on the cathode side of explants toward the cathode, but prevented the migration of cells on the anode side of explants toward anode. EFs also increased the speed of cells on the cathode side of the explants migrating toward cathode (see FIG. 3B). Immunocytochemical labeling showed that 71% of the cells migrating toward the cathode are nestin- and DCX-positive cells (see FIGS. 4A-C). These data suggest that in some embodiments EFs may act as a directional guidance cue to control and expedite NSPC migration toward cathode.

### Example 3

### EF-directed NSPC migration requires activation of N-methyl-D-aspartate receptors

To understand how EF-directed NSPC migration is triggered at the cellular level, the molecular signals that might be responsible for EF-directed NSPC migration were examined. As important membrane proteins, NMDARs have been shown to play a key role in regulating neural migration by affecting Ca²⁺ transient frequency migration. The following experiments were set up to determine whether NMDARs are involved in EF-directed NSPC migration, and whether the downstream signals can mediate the effect of NMDARs.

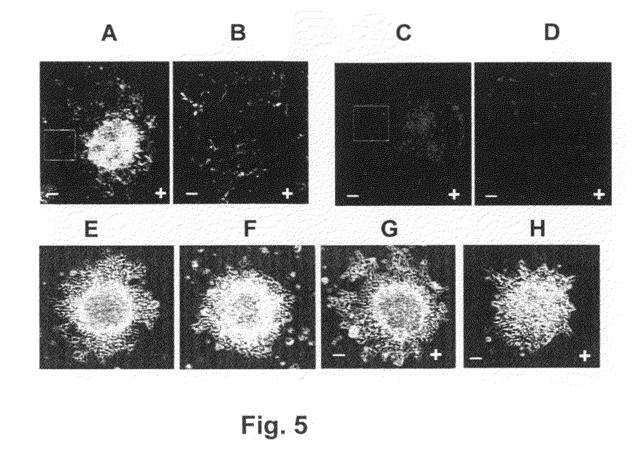

To determine whether NMDARs are expressed in NSPCs, immunocytochemical staining showed that the NR1 and NR2B, but not NR2A (data not shown), subunits of NMDARs were expressed in the majority (87%) of the cells migrating towards the cathode (FIGS. 5A-D), suggesting that NR2B-containing NMDARs may play a major role in mediating NMDAR function in NSPCs. The effects of DAPV, a selective NMDAR antagonist, on NSPC migration in the explant cultures were examined. It was found that DAPV (10 μM) significantly inhibited NSPC migration toward cathode on the cathode side of explants in a small EF (30 mV/mm treatment for 10 hours; FIGS. 5E-G and 6). These data indicate that activation of NMDARs by EF stimulation mediates EF-induced NSPC migration, suggesting that altered activity of membrane proteins may be a critical first step for a migrating cell to respond to EF stimulation.

### Example 4

### EF Stimulation Enhances a Physical Association of NMDARs With the Activator of Rac1

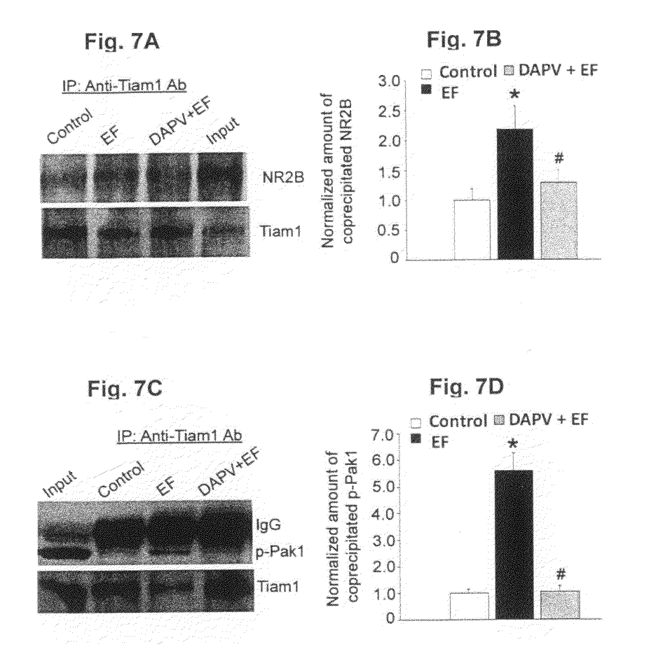

Rearrangement of the actin cytoskeletal network is an essential process in neural migration. Recent evidence indicates that the Rho GTPase Rac1 plays an important role in mediating neural migration through regulating actin cytoskeletal remodeling. If NMDARs are required for EF-induced NSPC migration, an intracellular signal pathway would link NMDARs to actin cytoskeletal remodeling. Not to be bounded by theory, it is hypothesized that in some embodiments the activated NMDARs by EF stimulation might mediate NSPC migration through interacting with the Rac1-dependent signal transduction pathway. To address this possibility, we performed coimmunoprecipitation assays to examine whether EFs could increase a coupling of NMDARs to the guanine nucleotide exchange factor TIAM1 (The invasion inducing T-lymphoma and metastasis 1), a specific Rac1 activator that causes Rac1 activation and subsequent actin polymerization. Immunoprecipitation with an anti-TIAM1 antibody resulted in coprecipitation of NMDARs in control explants, suggesting a physical interaction between NMDARs and TIAM1 in biological conditions. Interestingly, our data showed that treatment of explants with physiological EFs at strength of 250 mV/mm for 60 minutes significantly increased the association of NMDARs with TIAM1 (FIGS. 7A-B). Importantly, we demonstrated that the NMDAR antagonist DAPV (10 μM) inhibited the EF-induced increase of NMDAR association with TIAM1 (FIGS. 7A-B). These data indicate that EF stimulation can activate NMDARs on the cell membrane, which leads to an increased association of NMDARs with Rac1 activator TIAM1. Thus, through forming a complex with Rac1-associated signals, NMDARs may transmit extracellular EF stimulation to the intracellular Rac1 signaling transduction pathway and thereby mediate EF-directed NSPC migration.

To obtain further evidence to support the interaction of Rac1 signals with NMDARs in the involvement of EF-induced NSPC migration, we examined whether p21-activated kinase 1 (PAK1), a downstream target of Rac1 for actin polymerization, was involved in EF-induced interaction between NMDARs and TIAM1. By performing coimmunoprecipitation assays, we showed that there was an increased association of the phosphorylated PAK1 (p-PAK1, i.e., the activated form of PAK1) with TIAM1 in EF-exposed explants, and that inhibition of NMDARs by DAPV abolished the enhanced association of p-PAK1 with TIAM1 (FIGS. 7C-D). These data suggest that in some embodiments EF stimulation may lead to the recruitment of activated PAK1 to the NMDAR-TIAM1 complex and this protein-protein interaction process requires NMDAR activation.

### Example 5

### EF-Induced NMDAR Activation Leads to Increased Activity of Rac1 Signal Pathway

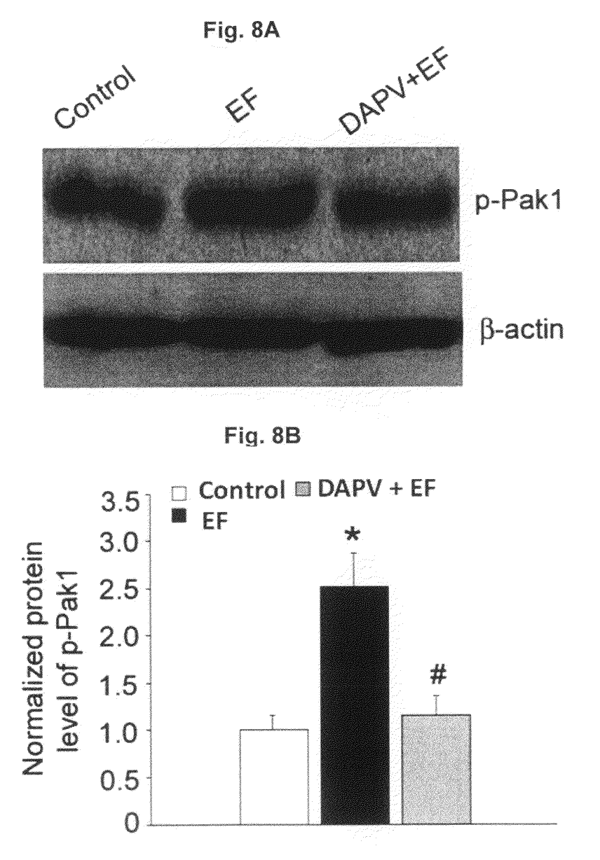

The observed formation of NMDAR/TIAM1/p-PAK1 protein complex in an EF suggests that the activity of PAK1 may be enhanced due to the activation of the NMDAR/TIAM1/p-PAK1 signal cascade. Because increased activation of PAK1 represents an enhanced activity of Rac1 signal pathway, we set up to determine whether the phosphorylation levels of PAK1 are altered by EF stimulation. Using an antibody against p-PAK1 at serine 423 in immunoblot assays, we found that EF treatment (250 mV/mm) for 60 min significantly enhanced PAK1 phosphorylation (FIGS. 8A-B), indicating an increased activity of PAK1 by EF stimulation. Moreover, treatment with the NMDAR antagonist DAPV significantly attenuated the EF-mediated increase of PAK1 phosphorylation (FIGS. 8A-B), suggesting that NMDAR activation contributes to EF-induced increase of PAK1 activity. These data suggest that EF stimulation, via activation of NMDARs, may promote a physical association of NMDARs with Rac1-associated signals and thereby enhance the activity of Rac1-dependent signal transduction pathway. Thus, these results support the possibility that NMDAR/TIAM1/Rac1/PAK1 pathway may play a crucial role in mediating EF-induced NSPC migration.

### Example 6

### EF-Induced NMDAR Activation Promotes Association of TIAM1 With Actin Cytoskeleton

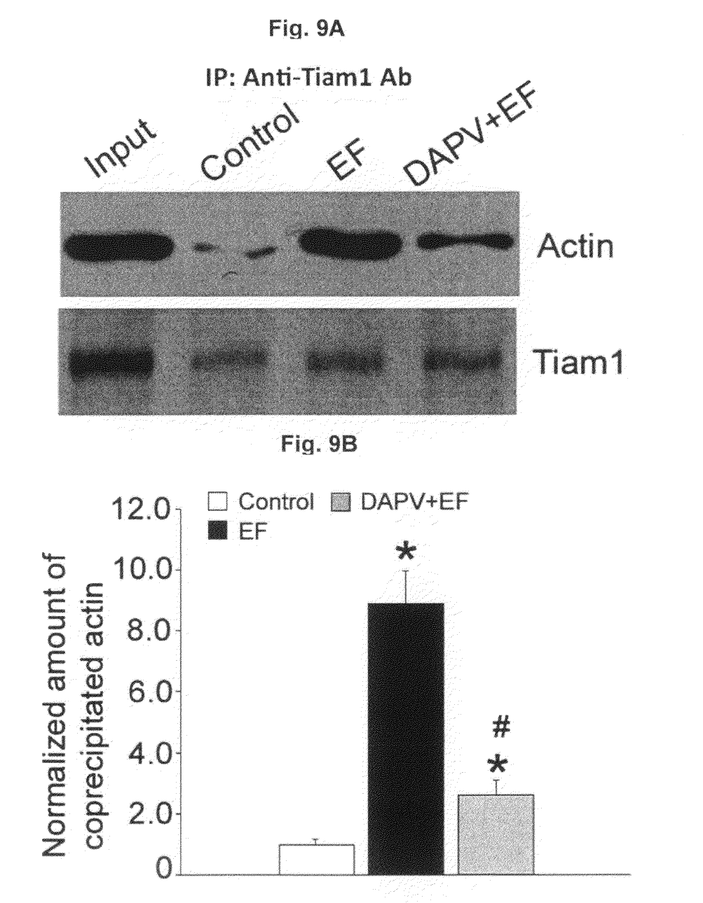

If the activated NMDAR/TIAM1/Rac1/PAK1 signal pathway is responsible for the EF-induced NSPC migration, the actin cytoskeleton should respond to Rac1 signaling that is known to couple to the actin cytoskeletal remodeling process to mediate cell migration. To determine whether there was an EF-induced interaction between Rac1 signals and the actin cytoskeleton, coimmunoprecipitation assays using protein from control and EF-exposed explants were performed. It was found that anti-TIAM1 antibody led to the coprecipitation of actin in the control explants (FIGS. 9A-B) and the coprecipitated amount of actin protein was significantly increased in EF-exposed explants (FIGS. 9A-B). It was also found that in some embodiments DAPV treatment suppressed EF-induced increase of association between actin and TIAM1 (FIGS. 9A-B). These data suggest that in some embodiments EF-controlled NSPC migration may be mediated by a physical interaction of NMDAR/TIAM1/Rac1/PAK1 signal complex with the actin cytoskeleton and that this interaction is dependent on NMDAR activation.

The data disclosed herein show that NMDARs may be activated by EF stimulation and activation of NMDARs may lead to an increase of physical association of these channels with Rac1, activator TIAM1 and effector PAK1, and subsequently an enhancement of association with actin cytoskeleton. These data also suggest that NMDAR may act as a membrane transducer to couple the extracellular EF stimulation to the intracellular TIAM1/Rac1/PAK1/actin pathway and thus play a role in mediating NSPC migration. The protein complex NMDAR/TIAM1/Rac1/PAK1/actin may act as a novel signal pathway in the EF-exposed Migrating NSPCs. Further, the protein-protein interactions of NMDARs with Rac1 signals and actin cytoskeleton may represent a general cellular and molecular mechanism underlying NMDAR-mediated neural migration in the CNS.

All publications and patent applications mentioned in this specification are herein incorporated by reference to the same extent as if each individual publication or patent application was specifically and individually indicated to be incorporated by reference. It is to be understood that this disclosure has been described in detailed by way of illustration and example in order to acquaint others skilled in the art with the invention, its principles, and its practical application. Further, the specific embodiments provided herein as set forth are not intended to be exhaustive or to limit the disclosure, and that many alternatives, modifications, and variations will be apparent to those skilled in the art in light of the foregoing examples and detailed description. Accordingly, this disclosure is intended to embrace all such alternatives, modifications, and variations that fall within the spirit and scope of the following claims. While some of the examples and descriptions above include some conclusions about the way the methods may function, the inventors do not intend to be bound by those conclusions and functions, but put them forth only as possible explanations in light of current understanding.

While the invention has been described with respect to a limited number of embodiments, the specific features of one embodiment should not be attributed to other embodiments of the invention. No single embodiment is representative of all aspects of the invention. In some embodiments, the methods may include numerous compounds or steps not mentioned herein. In other embodiments, the methods do not include, or are substantially free of, any steps not enumerated herein. Variations and modifications from the described embodiments exist. It is noted that the methods of repairing, treating, managing or preventing a brain damage in a brain of a mammal, such as human, are described with reference to a number of steps. These steps can be practiced in any sequence. One or more steps may be omitted or combined but still achieve substantially the same results. The appended claims intend to cover all such variations and modifications as falling within the scope of the invention.

All publications and patent applications mentioned in this specification are herein incorporated by reference to the same extent as if each individual publication or patent application was specifically and individually indicated to be incorporated by reference.

What is claimed is:

1. A method of repairing, treating, managing or preventing a brain damage in a brain of a mammal, the method comprising any one or more steps as follows:
a). applying a direct current electric field to direct or modulate the migration of one or more neural stem cells or progenitor cells towards at least a portion of the region of the brain damage;
b). administering an electric field between a cerebral ventricle and the meninx, inclusive, of the brain;
c). activating a membrane protein of neural stem cell or progenitor cell by a direct current electric field;
d). applying an electric field to promote neurogenesis in the subventricular zone or subgranular zone of the brain.

2. The method of claim 1, wherein the direct current electric field is between a cathode and an anode.

3. The method of claim 2, wherein the cathode is at or near the region of the brain damage or at the skull near the region of the brain damage.

4. The method of any of claims 2, wherein the anode is placed at the subventricular zone, at the subgranular zone, or the inside of a mouth or nasal opening.

5. The method of claim 1, wherein the electric field is a direct current electric field, a pulsed direct current electric field, an alternative current electric field, a capacitatively coupled electric field (CCEF), or an electric field induced by a pulsed magnetic field.

6. The method of claim 1, wherein the electric field is a direct current electric field between an anode at or near the cerebral ventricle and a cathode at or near the meninx.

7. The method of claim 6, wherein the direct current is provided by a power system comprising a battery and a resistor.

8. The method of any of claims 1, wherein the meninx is the dura mater, arachnoid mater or pia mater of the damaged brain.

9. The method of any of claims 1, wherein the cerebral ventricle is a lateral ventricle, the third ventricle or the fourth ventricle of the damaged brain.

10. The method of claim 1, wherein the electric field is a direct current electric field between an anode at or near the cerebral ventricle and a cathode at the skull near the region of the brain damage.

11. The method of any of claims 1, wherein the neural stem cells or progenitor cells migrate from the subventricular zone to the region of the brain damage.

12. The method of any of claims 1, wherein the neural stem cells or progenitor cells migrate from the subgranular zone to the region of the brain damage.

13. The method of claim 1, wherein the direct current is provided by a power system comprising a battery and a resistor.

14. The method of claim 13, wherein the battery has a voltage from about 0.1 volts to about 36 volts.

15. The method of claim 13, wherein the resistor has an electrical resistance from about 1 ohm to about 100 megaohms.

16. The method of claim 1, wherein the brain damage is a traumatic brain injury, non-traumatic brain injury or neurodegenerative disease, preferably is a non-traumatic brain injury or a neurodegenerative disease.

17. The method of claim 16, wherein the non-traumatic brain injury is stroke, meningitis, hypoxia or anoxia.

18. The method of claim 16, wherein the neurodegenerative disease is Alexander's disease, Alper's disease, Alzheimer's disease, Amyotrophic lateral sclerosis, Ataxia telangiectasia, Batten disease, Bovine spongiform encephalopathy, Canavan disease, Cockayne syndrome, Corticobasal degeneration, Creutzfeldt-Jakob disease, Huntington's disease, AIDS dementia complex, Kennedy's disease, Krabbe's disease, dementia with lewy bodies, Machado-Joseph disease, Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Lichtheim's disease, Schizophrenia, Spinocerebellar ataxia, Spinal muscular atrophy, Steele-Richardson-Olszewski disease or Tabes dorsalis.

19. The method of any of claims 1, wherein the magnitude of the direct current varies in time.

20. The method of any of claims 1 further comprising applying a second electric field to direct or modulate the migration of one or more neural stem cells or progenitor cells towards at least a portion of the region of the brain damage.

21. The method of claim 20, wherein the second electric field is a direct current electric field or an alternative current electric field.

22. The method of any of claims 1 further comprising applying a pulsed magnetic field to the region of the brain damage.

23. The method of claim 1, wherein the mammal is a human.
